(19) United States
(12) Patent Application Publication
(10) Pub. No.: US 2008/0243180 A1
(43) Pub. Date: Oct. 2, 2008

(54) SURGERY FIXATION FORCEPS

(76) Inventor: YEN-YUE LIN, Taipei (TW)

Correspondence Address: LEONG C LEI, PMB # 1008, 1867 YGNACIO VALLEY ROAD, WALNUT CREEK, CA, 94598, US

(21) Appl. No.: 11/970,536

(22) Filed: Jan. 8, 2008

Related U.S. Application Data
(63) Continuation-in-part of application No. 11/042,131, filed on Jan. 26, 2005, now abandoned.

(51) Int. Cl. A61B 17/50; A61B 17/00; A61B 17/32

(52) U.S. Cl. 606/211; 606/205; 606/167

(57) ABSTRACT

A pair of surgery fixation forceps which includes a pair of tweezers having an upper end, a blade holder including a U-shaped base and a rod portion, the rod portion being formed with two grooves, the U-shaped base having two arms, an adjusting screw for securing the blade holder to the tweezers; and a cutting blade having an elongated slot which includes a first engaging portion and a second engaging portion which is narrower than the first engaging portion, the two opposite sides of the second engaging portion being fitted into the grooves of the rod portion of the blade holder, the first engaging portion being configured to be snugly fitted with the rod portion of the blade holder thereby enabling the cutting blade to be secured to the blade holder, the cutting blade having two ears at two upper corners thereof and a knife edge therebetween.

### CROSS-REFERENCE TO RELATED APPLICATION

This is a continuation-in-part of the co-pending patent application Ser. No. 11/042,131, owned by the same applicant.

### BACKGROUND OF THE INVENTION

1. (a) Technical Field of the Invention

This invention is related to a pair of surgery fixation forceps and in particular to one which can be used as a pair of ligature cutting scissors in suture operation to cut suture lines.

2. (b) Description of the Prior Art

Referring to FIGS. 8A and 8B, the doctor uses her or his one hand holding a pair of tweezers 8 to hold the peripheral skin to a wound 9 and the other hand holding a pair of ligature forceps 7. The ligature forceps 7 are used to hold a needle 71 which is connected with a suture 72, so that the doctor can completes the suture operation for the patient by stitching. However, upon completing each stitch, the suture 72 must be cut off using a pair of ligature cutting scissors 6 before proceeding to next stitching. Therefore, upon completing a stitch, the doctor has to spare one hand to fetch for the ligature cutting scissors 6 in order to cut off the suture, or a nurse must be available to do the cutting for the doctor making the entire suture operation not meeting economic benefits in either way.

### SUMMARY OF THE INVENTION

This invention is related to a pair of surgery fixation forceps which can be used as a pair of ligature cutting scissors in suture operation.

It is the primary object of the present invention to provide a pair of surgery fixation forceps which includes a pair of tweezers having an upper end, a blade holder including a U-shaped base and a rod portion extending upwardly from an arm of the U-shaped base, the rod portion being formed with two grooves at two opposite sides thereof, the U-shaped base having two arms between which there is an opening, the opening 23 is dimensioned to fit over the upper end of the tweezers, an adjusting screw extending through a hole of the U-shaped base to secure the blade holder to the tweezers; and a cutting blade having an elongated slot which includes a first engaging portion and a second engaging portion which is narrower than the first engaging portion, the second engaging portion being dimensioned so that the two opposite sides of the second engaging portion are fitted into the grooves of the rod portion of the blade holder, the first engaging portion being configured to be snugly fitted with the rod portion of the blade holder thereby enabling the cutting blade to be secured to the blade holder, the cutting blade having two ears at two upper corners thereof and a recess between the two ears, the recess being provided a knife edge for cutting off a suture, whereby the surgery fixation forceps can be used as a pair of ligature cutting scissors in suture operation to cut suture lines.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of a pair of surgery fixation forceps according to a first embodiment of the present invention;

FIG. 2 is a perspective view of the surgery fixation forceps according to tie first embodiment of the present invention;

FIG. 2A is an enlarged sectional view of a portion of FIG. 2;

FIG. 3 is an exploded view of a pair of surgery fixation forceps according to a second preferred embodiment of the present invention;

FIG. 3A is an enlarged view of a portion of FIG. 3;

FIG. 4 is a perspective view of the surgery fixation forceps according to the second preferred embodiment of the present invention;

FIG. 4A is an enlarged view of a portion of FIG. 4;

FIG. 5 is a perspective view of the surgery fixation forceps according to a third preferred embodiment of the present invention;

FIG. 6 is a working view of the surgery fixation forceps according to the first embodiment of the present invention;

FIG. 7 is a working view of the surgery fixation forceps according to the second embodiment of the present invention; and

FIGS. 8A and 8B are schematic views showing multiple appliances are used in a suture operation.

### DETAILED DESCRIPTION OF THE PREFERRED EMBODIMENT

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

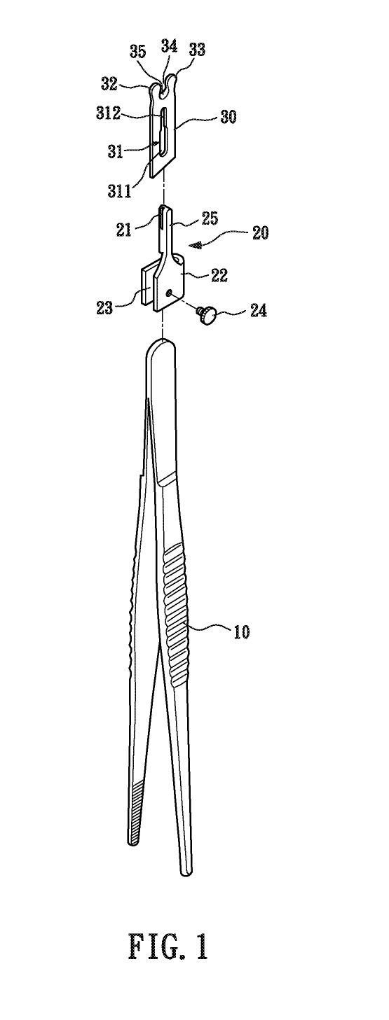

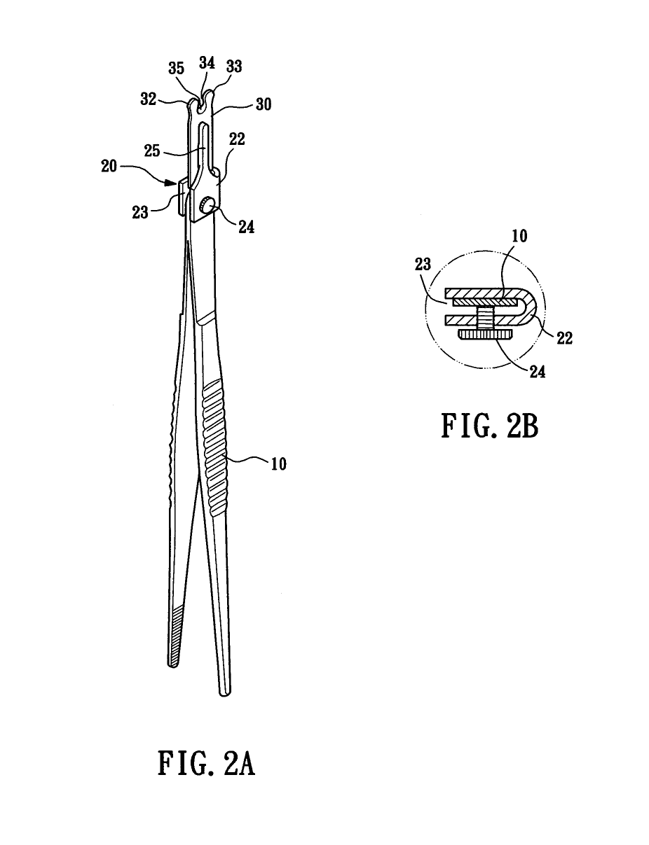

With reference to the drawings and in particular to FIGS. 1, 2 and 2A, the surgery fixation forceps according to the present invention comprises a pair of tweezers 10, a blade holder 20, and a cutting blade 30. The tweezers 10, the blade holder 20 and the cutting blade 30 are assembled to form the surgery fixation forceps which can be used as a pair of tweezers or a pair of ligature of cutting scissors as required thereby enabling a doctor to cut suture lines as soon as the suture operation is finished.

The tweezers 10 is conventional in structure. The blade holder 20 includes a U-shaped base 22 and a rod portion 25 extending upwardly from an arm of the U-shaped base 22. The rod portion 25 is formed with two grooves 21 at two opposite sides thereof. The U-shaped base 22 has two arms between which there is an opening 23. The opening 23 is dimensioned to fit over the upper end of the tweezers 10. An adjusting screw 24 extends through a hole of the U-shaped base 22 to secure the blade holder 20 to the tweezers 10.

The cutting blade 30 has an elongated slot 31 which includes a first engaging portion 311 and a second engaging portion 312 which is narrower than the first engaging portion 311. The second engaging portion 312 is dimensioned so that the two opposite sides of the second engaging portion 312 are fitted into the grooves 21 of the rod portion 25 of the blade holder 20. The cutting blade 30 is resilient and the first engaging portion 311 is configured to be snugly fitted with the rod portion 25 of the blade holder 20 thereby enabling the cutting blade 30 to be secured to the blade holder 20. The cutting blade 30 is provided with two circular ears 32 and 33 at two upper corners thereof. Between the two circular ears 32 and 33 there is a recess 34 formed with a knife edge 35 which can be used as a ligature cutting scissors for cutting off the free end of a suture 72.

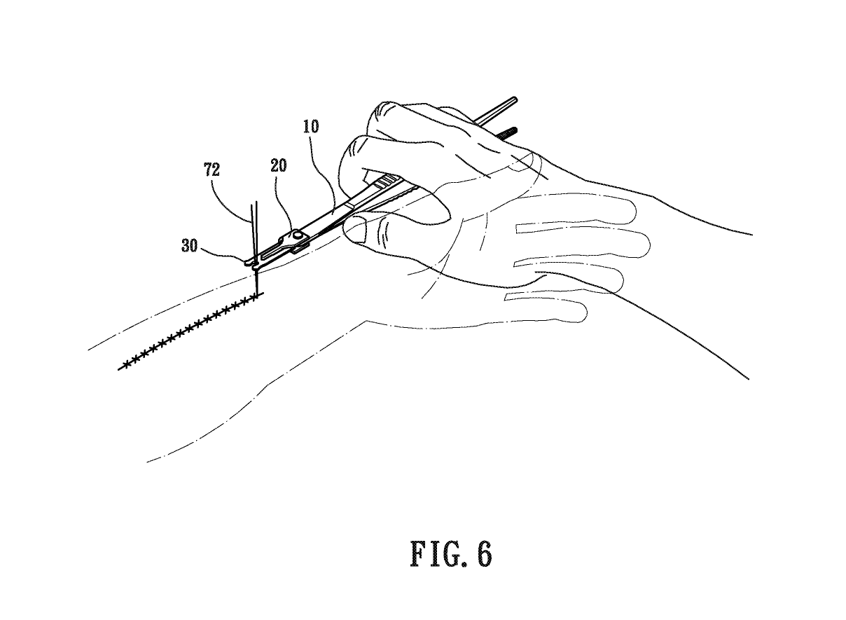

FIG. 6 is a working view of the surgery fixation forceps according to the first embodiment of the present invention. As shown, the surgery fixation forceps are used as a pair of ligature cutting scissors which are pushed forward to cut off the free end of a suture 72 which is tightened by an appropriate tool (not shown). The tweezers 10, blade holder 20 and cutting blade 30 can be easily disengaged from one another thus making it convenient to be replaced as required.

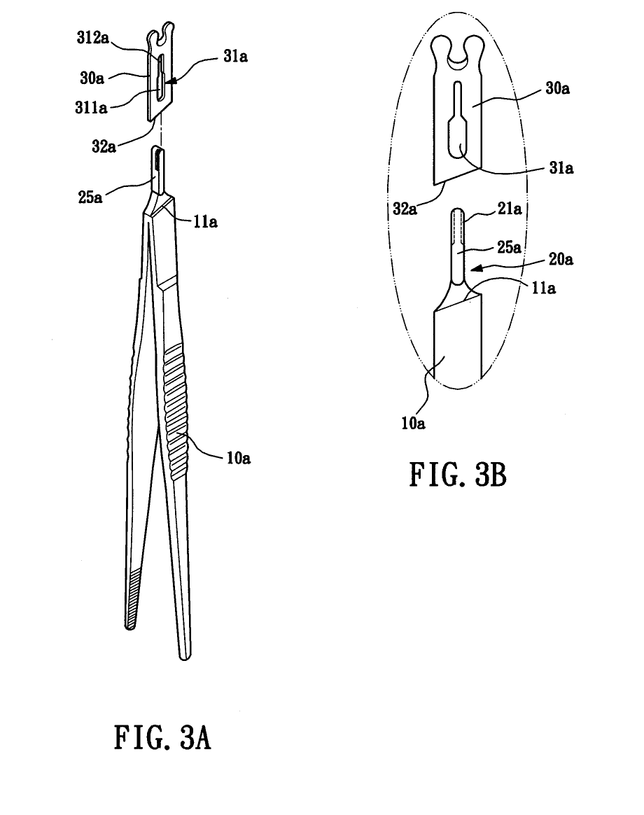

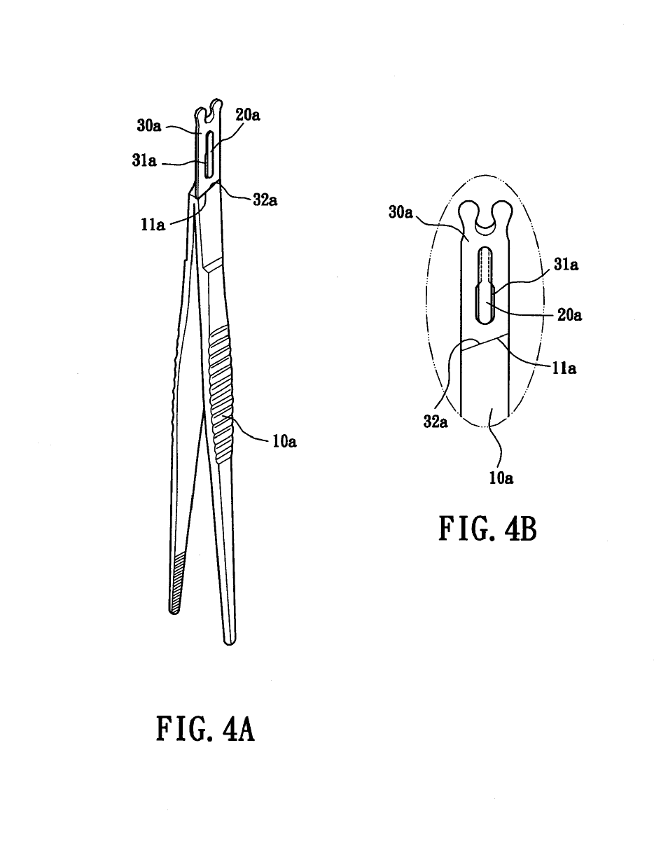

FIGS. 3, 3A, 4 and 4A illustrate a pair of surgery fixation forceps according to a second preferred embodiment of the present invention. As can be seen, the surgery fixation forceps comprise a pair of tweezers 10a, a blade holder 20a which is integrally formed with an upper end of the tweezers 10a, and a cutting blade 30. The blade holder 20 includes an upwardly extending rod portion 25a which is formed with two grooves 21a at two opposite sides thereof. The cutting blade 30a has an elongated slot 31a which includes a first engaging portion 311a and a second engaging portion 312a which is narrower than the first engaging portion 311a. The second engaging portion 312a is dimensioned so that the two opposite sides of the second engaging portion 312a are fitted into the grooves 21 of the rod portion 25a of the blade holder 20a. The first engaging portion 311a is dimensioned to receive the rod portion 25a of the blade holder 20a thereby enabling the cutting blade to be secured to the blade holder 20a. The cutting blade 30a is provided with two circular ears 32a and 33a at two upper corners thereof. Between the two circular ears 32a and 33a there is a recess 34a formed with a knife edge 35a. The upper end of the tweezers 10a has a shoulder 11a which bears against the bottom edge 32a of the cutting blade 30a for positioning the cutting blade 30a.

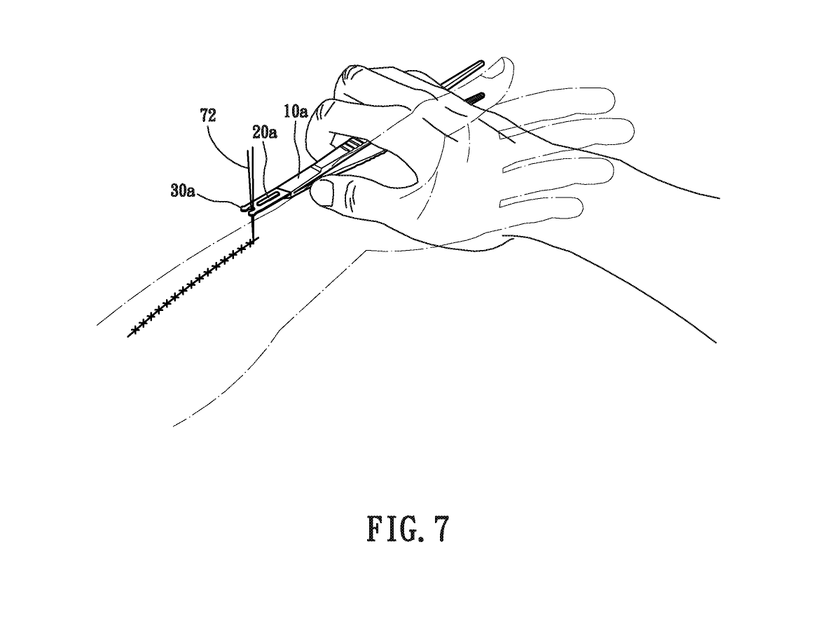

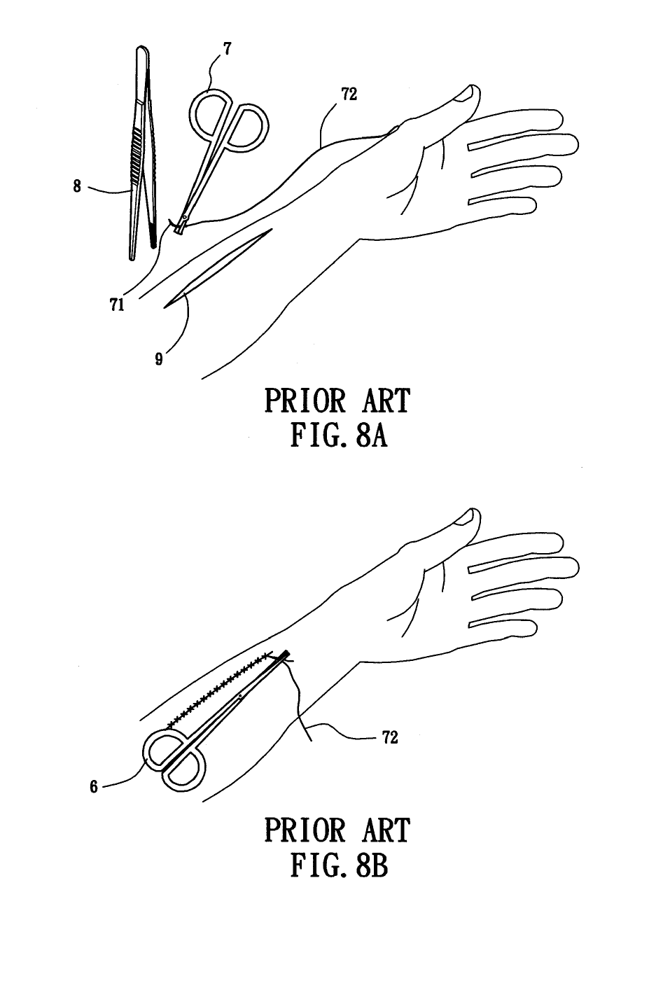

FIG. 7 is a working view of the surgery fixation forceps according to the second embodiment of the present invention. As illustrated, the surgery fixation forceps are used as a pair of ligature cutting scissors which are pushed forward to cut off the free end of a suture 72 which is tightened by an appropriate tool (not shown). The tweezers 10a, blade holder 20a and cutting blade 30a can be easily disengaged from one another thus making it convenient to be replaced as required.

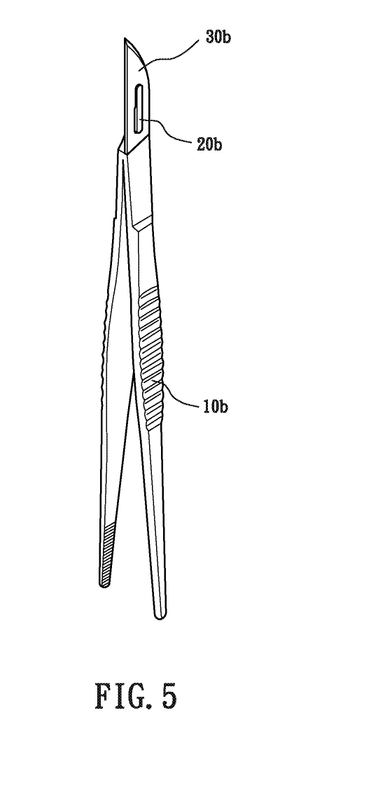

FIG. 5 is a perspective view of the surgery fixation forceps according to a third preferred embodiment of the present invention. As shown, the surgery fixation forceps comprise a pair of tweezers 10b, a blade holder 20b which is integrally formed with an upper end of the tweezers 10, and a cutting blade 30b. The cutting blade 30b is provided with a curved knife edge at one side thereof.

In conclusion, a doctor can use the surgery fixation forceps according to the present invention as a pair of tweezers or a pair of ligature scissors as required thereby making it possible form him to cut off the suture without changing the surgery fixation forceps to a pair of ligature scissors or the help of an assistant. Further, the present invention has at the least the following advantages:

1. Shortening the time required for suture and anesthesia and reducing anesthetic dosage;

2. Fewer nurses are required to attend the operation;

3. Reducing use or replacement of surgery appliances; and

4. Easy to dismantle thereby making it convenient for replacement or stowage.

The cutting blade has two circular ears at two upper corners between which there is a knife edge. The cutting blade has at least the following advantages:

1. Easy to cut off a suture;

2. Causing no damage to the patient or skin;

3. Suitable for use in subcutaneous.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A pair of surgery fixation forceps comprising:
a pair of tweezers having an upper end;
a blade holder including a U-shaped base and a rod portion extending upwardly from an arm of said U-shaped base, said rod portion being formed with two grooves at two opposite sides thereof, said U-shaped base having two arms between which there is an opening, said opening being dimensioned to fit over said upper end of the tweezers;
an adjusting screw extending through a hole of said U-shaped base to secure said blade holder to said tweezers; and
a cutting blade having an elongated slot which includes a first engaging portion and a second engaging portion which is narrower than said first engaging portion, said second engaging portion being dimensioned so that said two opposite sides of said second engaging portion are fitted into said grooves of said rod portion of said blade holder, said first engaging portion being configured to be snugly fitted with said rod portion of said blade holder thereby enabling said cutting blade to be secured to said blade holder, said cutting blade having two ears at two upper corners thereof and a recess between said two ears, said recess being provided a knife edge for cutting off a suture.

2. The surgery fixation forceps as claimed in claim 1, wherein said ears are circular in shape.
